(19) United States
(12) Patent Application Publication
(10) Pub. No.: US 2020/0054875 A1
(43) Pub. Date: Feb. 20, 2020

(54) SHARKLET TOPOGRAPHIES TO CONTROL NEUTRAL CELL INTERACTIONS WITH IMPLANTED ELECTRODES

(71) Applicant: UNIVERSITY OF FLORIDA RESEARCH FOUNDATION, INCORPORATED, Grainesville, FL (US)

(72) Inventors: Sahba MOBINI, Gainesville, FL (US); Christine E. SCHMIDT, Gainesville, FL (US); Anthony B. BRENNAN, Gainesville, FL (US); Cary A. KULIASHA, Gainesville, FL (US); Jack JUDY, Gainesville, FL (US)

(21) Appl. No.: 16/485,685

(22) PCT Filed: Feb. 13, 2018
(86) PCT No.: PCT/IB2018/050886

§ 371 Date: Aug. 13, 2019

Related U.S. Application Data
(60) Provisional application No. 62/458,064, filed on Feb. 13, 2017.
(60) Provisional application No. 62/629,312, filed on Feb. 12, 2018.

(51) Int. Cl. A61N 1/36; A61N 1/05; C12N 5/079; A61L 27/34

(52) U.S. Cl. CPC A61N 1/3605; A61N 1/0529; A61N 1/0551; C12N 5/0622; A61L 2430/32; C12N 2533/54; C12N 2533/74; C12N 2535/00; A61L 27/34

(57) ABSTRACT

Tissue-engineered nerve scaffolds, articles for up-selecting desired cell proliferation and down-selecting undesired cell proliferation, and methods of manufacturing the same are provided. The tissue-engineered nerve scaffold includes a hydrogel having a surface. The surface has a topography including a micropattern defined by a plurality of spaced features attached to or projected into the hydrogel. The micropattern facilitates attachment and alignment of neural cells and reduces attachment and alignment of cells associated with scar-tissue formation and encapsulation.

### CROSS-REFERENCE TO RELATED APPLICATIONS

This application claims priority to U.S. Patent Application No. 62/458,064 filed Feb. 13, 2017, and U.S. Patent Application No. 62/629,312 filed Feb. 12, 2018, which are incorporated by reference herein in their entirety.

### STATEMENT REGARDING FEDERALLY SPONSORED RESEARCH OR DEVELOPMENT

This invention was made with government support under HR0011-15-2-0030 awarded by Department of Defense/DARPA. The government has certain rights in the invention.

### TECHNICAL FIELD

The presently-disclosed invention relates generally to patterns for up-selecting desired cell proliferation and down-selecting undesired cell proliferation, and more particularly to tissue-engineered nerve scaffolds, articles for up-selecting desired cell proliferation and down-selecting undesired cell proliferation, and methods of manufacturing the same.

### BACKGROUND

Nerve-scaffold technology has advanced to the point where patients with severe nerve damage, who might otherwise suffer from chronic, stabbing, radiating, and debilitating pain, numbness, loss of sensation, and partial or full loss of limb movement, are now able to recover function. However, such nerve-scaffold technology has not yet been used to serve patients for which limb amputation is unavoidable.

To solve this problem a tissue-engineered electronic nerve interface (TEENI) was developed. In the TEENI device, multi-electrode “threads” are embedded into tissue-engineered hydrogel nerve scaffolds to enable natural nerve regeneration into the scaffold. The threads have multiple electrodes spaced densely along their length and multiple parallel threads are distributed throughout the axial cross section of the tissue-engineered scaffold. By having the tissue regenerate around the electrodes, the physical constraints and consequences associated with requiring the electrode interface to penetrate the nerve are avoided. Unlike other approaches that use electrode probes that are always rigid (e.g., USEAS,), probes that are rigid pre-implant and then compliant after implant (shape-memory polymers,), or always compliant but pulled through the nerve behind a rigid needle (e.g., TIME,), the multi-electrode interfaces used in the TEENI never have to be rigid or pushed through anything. As a result they can be engineered to be as mechanically compliant as possible, such being formed in a serpentine geometry that allows for a far greater structure compliance than possible with a straight geometry, but does not require a rigid dissolvable coating designed to again facilitate implantation (e.g., carboxymethylcellulose).

Despite the TEENI thread's narrow geometry and extreme mechanical compliance, fundamentally it is still made of materials foreign to the body. Thus, although the TEENI threads are placed inside the TEENI scaffold, hidden away from the wound-healing cascade, eventually, as the hydrogel interior is remodeled during nerve regeneration, the TEENI thread is revealed to the regenerated nerve and thus could set off a foreign-body tissue response, albeit much delayed and perhaps muted. Selective cell control is required to achieve optimum biocompatibility and integration of an implanted biomaterial to surrounding tissue. In the case of peripheral nerve regeneration, migration and differentiation of Schwann cells are crucial to robust axonal regeneration while fibrotic encapsulation of implanted devices, including microelectrodes and nerve interphases, by fibroblasts can reduce device efficacy leading to failure or rejection.

Accordingly, there still exists a need for a tissue-engineered nerve scaffold that prevents the foreign-body tissue response to the TEENI device.

### BRIEF SUMMARY OF THE INVENTION

One or more embodiments of the invention may address one or more of the aforementioned problems. Certain embodiments provide tissue-engineered nerve scaffolds, methods of manufacturing tissue-engineered nerve scaffolds, articles for up-selecting desired cell proliferation and down-selecting undesired cell proliferation, and methods of manufacturing articles for up-selecting desired cell proliferation and down-selecting undesired cell proliferation. In one aspect, a tissue-engineered nerve scaffold configured to coat a multielectrode neural interface is provided. The tissue-engineered nerve scaffold may include a hydrogel having a surface. The surface may have a topography comprising a micropattern defined by a plurality of spaced features attached to or projected into the hydrogel. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature, and the spaced features within each of the groupings may be spaced apart as an average distance from about 10 nm to about 200 μm. The micropattern facilitates attachment and alignment of neural cells and reduces attachment and alignment of cells associated with scar-tissue formation and encapsulation.

In another aspect, a method of manufacturing a tissue-engineered nerve scaffold configured to coat a multielectrode neural interface is provided. The method may include providing a hydrogel having a surface and forming a topography comprising a micropattern defined by a plurality of spaced features onto or projected into the surface of the hydrogel. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature, and the spaced features within each of the groupings may be spaced apart as an average distance from about 10 nm to about 200 μm. The micropattern facilitates attachment and alignment of neural cells and reduces attachment and alignment of cells associated with scar-tissue formation and encapsulation.

In yet another aspect, an article for up-selecting desired cell proliferation and down-selecting undesired cell proliferation is provided. The article may include a surface. The surface may have a topography comprising a micropattern defined by a plurality of spaced features attached to or projected into the article. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature. The micropattern changes cell behavior and differentiates between cell types.

In still yet another aspect, a method of manufacturing an article having a surface is provided. The method may include providing the article and forming a topography comprising a micropattern defined by a plurality of spaced features onto or projected into the surface of the article. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature. The micropattern changes cell behavior and differentiates between cell types.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is an image of a tissue-engineered nerve scaffold coating three TEENI thread sets in accordance with certain embodiments of the invention;

FIG. 2 illustrates an example of a “Sharklet” micropattern comprising a plurality of raised surface features which project out from the surface of a base article in accordance with certain embodiments of the invention;

FIG. 3 illustrates the pattern geometry of the “Sharklet” micropattern in accordance with certain embodiments of the invention and the comparative channel micropattern;

FIG. 4 is a collection of optical images showing the “Sharklet” pattern matrix for several “Sharklet” micropatterns projecting from a surface at a height of 1.3 μm in accordance with certain embodiments of the invention;

FIG. 5 is a collection of SEM images showing the comparative channel pattern matrix for several channel micropatterns projecting from a surface at a height of 3 μm;

FIG. 6 is a collection of optical images showing the comparative channel pattern matrix for several channel micropatterns projecting from a surface at a height of 1.3 μm;

FIG. 7 is a collection of SEM images showing the “Sharklet” pattern matrix for several “Sharklet” micropatterns etched into a surface at a depth of 1.3 μm in accordance with certain embodiments of the invention;

FIG. 8 is a collection of SEM images showing the comparative channel pattern matrix for several channel micropatterns etched into a surface at a depth of 1.3 μm;

FIG. 9A illustrates rat fibroblast viability on shallow patterns relative to a smooth surface in accordance with certain embodiments of the invention;

FIG. 9B illustrates rat Schwann cell viability on shallow patterns relative to a smooth surface in accordance with certain embodiments of the invention;

FIG. 10A illustrates rat fibroblast viability on deeper patterns relative to a smooth surface in accordance with certain embodiments of the invention;

FIG. 10B illustrates rat Schwann cell viability on deeper patterns relative to a smooth surface in accordance with certain embodiments of the invention;

FIG. 11 is a schematic block diagram illustrating a method of manufacturing a tissue-engineered nerve scaffold in accordance with certain embodiments of the invention;

FIG. 12 is a schematic block diagram illustrating a method of manufacturing an article for up-selecting desired cell proliferation and down-selecting undesired cell proliferation in accordance with certain embodiments of the invention; and

FIG. 13 illustrates a method of manufacturing an article for up-selecting desired cell proliferation and down-selecting undesired cell proliferation in accordance with certain embodiments of the invention.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

As previously discussed herein, despite the TEENI thread's narrow geometry and extreme mechanical compliance, fundamentally it is still made of materials foreign to the body. Thus, although the TEENI threads are placed inside the TEENI scaffold, hidden away from the wound-healing cascade, eventually, as the hydrogel interior is remodeled during nerve regeneration, the TEENI thread is revealed to the regenerated nerve and thus could set off a foreign-body tissue response.

Through combined effort and ingenuity, the inventors have developed a surface topography that inhibits fibroblast attachment while stimulating Schwann cell attachment, guidance, and differentiation. This surface topography includes a “Sharklet” micropattern. Sharklet, inspired by the dermal denticles of sharkskin, is effective at modulating the fouling response of a wide range of fouling vectors including bacteria, marine organisms, and mammalian cells, and the inventors have determined that this micropattern can be modified to inhibit fibroblast attachment and proliferation while stimulating Schwann cell attachment and proliferation. Similarly, the inventors have also identified that modifying this surface topography may allow it to be used in conjunction with a variety of cell types.

As used herein, the terms “substantial” or “substantially”, unless otherwise directed, may encompass the whole amount as specified, according to certain embodiments of the invention, or largely but not the whole amount specified according to other embodiments of the invention.

As used herein, the term “biodegradable” refers to a material that is derived from natural or synthetic sources and that is capable of being degraded within the host organism. Following implantation, biodegradable materials should maintain their mechanical properties until the material is no longer required, at which point the material may be absorbed and excreted by the host organism.

I. Tissue-Engineered Nerve Scaffold

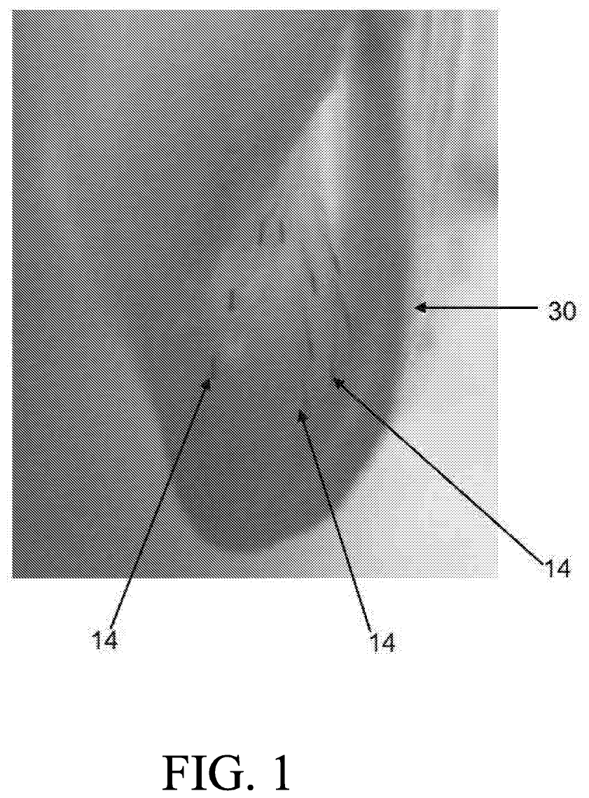

In accordance with certain embodiments, tissue-engineered nerve scaffolds configured to coat a multielectrode neural interface are provided. The tissue-engineered nerve scaffold includes a hydrogel having a surface. FIG. 1, for example, is an image of a tissue-engineered nerve scaffold coating a multielectrode neural interface in accordance with certain embodiments of the invention. As shown in FIG. 1, the hydrogel 30 surrounds three TEENI thread sets 14. As described in U.S. Patent Application No. 62/545,019, which is incorporated by reference in its entirety, the thread sets may comprise a plurality of spaced apart electronic leads that are encased within an insulating sheath, and the electronic leads may include one or more electrodes that are configured to come into contact with regenerated nerve fibers. In this regard, the TEENI thread sets functionally engage with a substantial portion of the peripheral nerve.

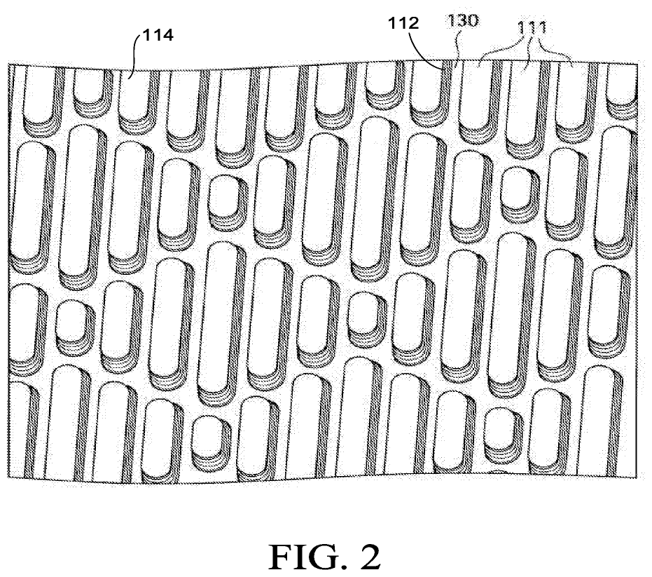

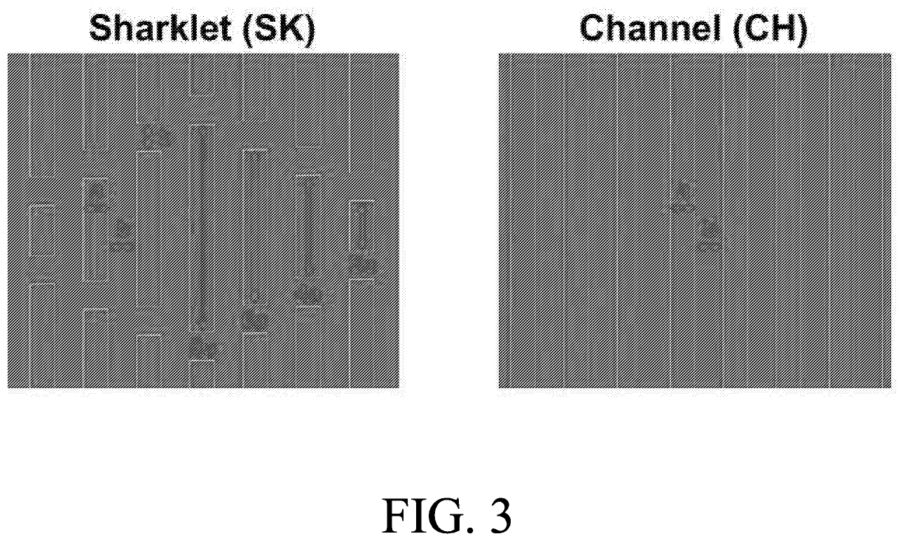

In accordance with certain embodiments, the surface of the hydrogel has a topography comprising a micropattern defined by a plurality of spaced features attached to or projected into the hydrogel. FIG. 2, for instance, illustrates an example of a “Sharklet” micropattern comprising a plurality of raised surface features 111, which each include a continuous sidewall 112 that projects outwardly from the surface 130 of a base article (e.g., the hydrogel described herein) in accordance with certain embodiments of the invention. As shown in FIG. 2, each spaced feature 111 comprises a surface 114 that is substantially parallel to a surface on a neighboring spaced feature and is a different length than a neighboring spaced feature. Although the surface 114 of each spaced feature 111 is shown as being substantially parallel to the surfaces of neighboring spaced features, the plurality of spaced features may have varying heights. Moreover, each spaced feature may comprise a rectangular oblong shape. For example, as shown in FIG. 2, each spaced feature may comprise a rectangular oblong shape having rounded edges. In contrast, as shown in FIG. 3, for instance, each spaced feature may comprise a rectangular oblong shape having straight edges.

According to certain embodiments, such as those described in detail herein, a single micropatterned hydrogel may surround the multielectrode neural interface. In such embodiments, for example, the micropatterned hydrogel may be bonded directly to one or more electrodes of the multielectrode neural interface.

In other embodiments, however, one or more of the electrodes of the multielectrode neural interface (i.e. TEENI electrodes) may comprise the micropattern instead of or in addition to the hydrogel. In this regard, in certain embodiments, one or more micropatterned electrodes may be surrounded by a patterned or unpatterned hydrogel.

In still further embodiments, the tissue-engineered nerve scaffold may comprise a second micropatterned hydrogel. For example, in some embodiments the first micropatterned hydrogel may be applied to polyimide, and the second micropatterned hydrogel may be cast upon the first micropatterned hydrogel.

The plurality of spaced features are spaced from each other to define an intermediate tortuous pathway and are arranged in a plurality of groupings. Neighboring groupings share a common feature, and the spaced features within each of the groupings are spaced apart at an average distance from about 10 nm to about 200 μm. In this regard, the micropattern facilitates attachment and alignment of neural cells and reduces attachment and alignment of cells associated with scar-tissue formation and encapsulation. Without intending to be bound by theory, it is believed that the micropattern is able to affect adhesion and proliferation of different cell types based on the way that the cells change the shape of their body and nuclei in interaction with the micropattern.

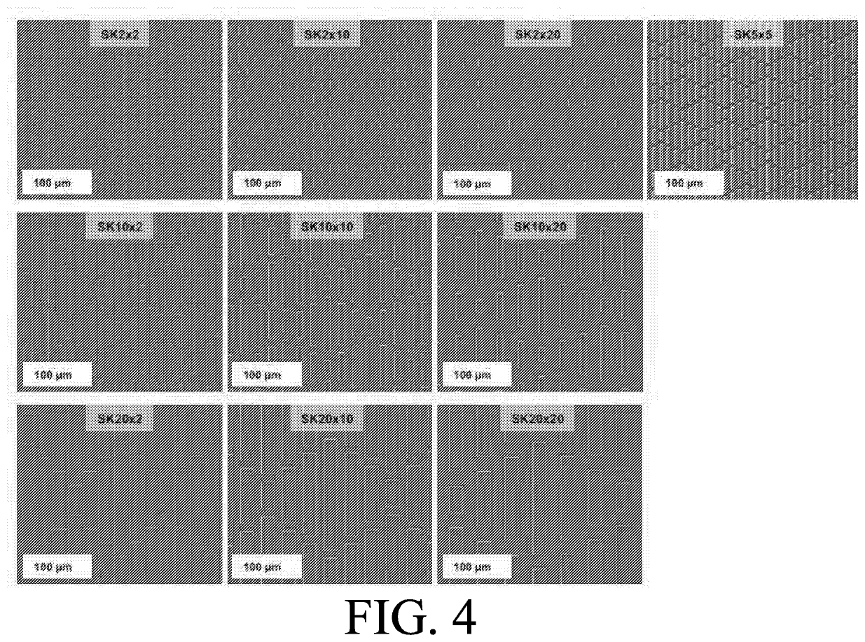

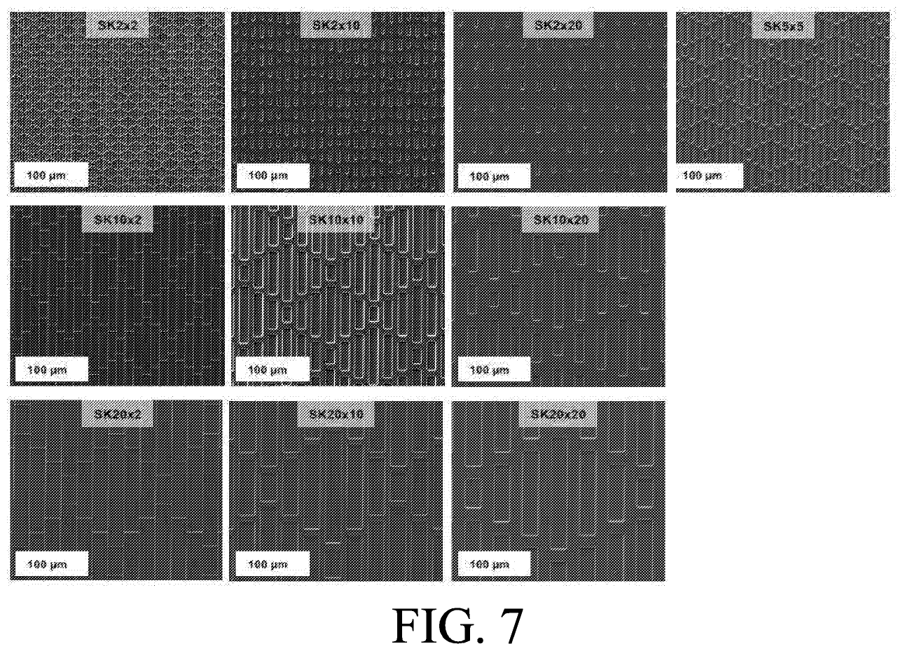

As previously mentioned herein, the plurality of spaced features may be attached to (and protrude from) or be projected into the hydrogel. FIG. 4, for example, is a collection of optical images showing the “Sharklet” pattern matrix for several “Sharklet” micropatterns protruding from a surface in accordance with certain embodiments of the invention. In contrast, FIG. 7, for instance, is a collection of SEM images showing the “Sharklet” pattern matrix for several “Sharklet” micropatterns etched into (and projecting into) a surface in accordance with certain embodiments of the invention. Either of these configurations are acceptable means of providing the plurality of spaced features onto or into a surface so long as the plurality of spaced features are at a different height than the surface such that the plurality of spaced features define a tortuous pathway among them.

In some embodiments, the micropattern comprises a plurality of spaced features that collectively define a plurality of diamond-shaped patterns (e.g., a rhombus shaped pattern) that are arranged end-to-end with adjacent diamond-shaped patterns. In this regard, FIG. 4 shows various micropatterns comprising a plurality of diamond-shaped patterns that are arranged in rows.

In particular, in the embodiment illustrated in FIG. 2, the micropattern includes a plurality of rows in which each row includes a plurality of diamond-shaped patterns. In some embodiments, adjacent diamond-shaped patterns within a row share a common spaced feature at the apex of each diamond-shaped pattern (see, e.g., reference character 2a in FIG. 3).

In certain embodiments, each row of diamond-shaped patterns is off-set relative to adjacent rows such that a spaced feature having the longest length (see, e.g., reference character 8a in FIG. 3) is aligned with a spaced feature in an adjacent row having the smallest length (see, e.g., reference character 2a in FIG. 3). Similarly, spaced features of intermediate lengths (see, e.g., reference characters 4a and 6a in FIG. 3) are aligned with spaced features of intermediate lengths of adjacent rows. In this way, each of the diamond-shaped patterns is off-set relative to adjacent diamond-shaped patterns in adjacent rows.

In accordance with certain embodiments, for example, each spaced feature may comprise a uniform width. In some embodiments, for instance, the spaced features within each of the groupings may be spaced apart vertically at a uniform average vertical distance. Similarly, the spaced features within each of the groupings may be spaced apart horizontally at a uniform average horizontal distance. Moreover, the uniform average vertical distance may be equal to the uniform average horizontal distance.

For example, in certain embodiments, each spaced feature may comprise a uniform width from about 2 μm to about 20 μm. In further embodiments, for instance, the spaced features within each of the groupings may be spaced apart at a uniform average horizontal distance from about 2 μm to about 20 μm. Similarly, the spaced features within each of the groupings may be spaced apart at a uniform average vertical distance that is equal to the uniform average horizontal distance, i.e. from about 2 μm to about 20 μm. As such, in certain embodiments, each of the uniform width, uniform average vertical distance, and uniform average horizontal distance may comprise at least about any of the following: 2, 4, 6, 8, 10, 12, 14, 16, 18, and 20 μm and/or at most about 20, 18, 16, 14, 12, 10, 8, 6, 4, and 2 μm (e.g., about 10-20 μm, about 2-10 μm, etc.). For example, each spaced feature may comprise a uniform width of about 20 μm, and the spaced features within each of the groupings may be spaced apart at a uniform average horizontal distance and a uniform average vertical distance of about 2 μm.

Moreover, as shown in FIG. 3, in some embodiments each spaced feature may comprise a length that is a multiple of the width of that feature. For example, feature 2a has a length that is twice its width, feature 4a has a length that is four times its width, feature 6a has a length that is six times its width, and feature 8a has a length that is eight times its width. Nevertheless, FIG. 3 is merely an example, and the length of the features may vary based on the selected micropattern.

As previously discussed herein, plurality of spaced features are spaced from each other to define an intermediate tortuous pathway. According to certain embodiments, for instance, the intermediate tortuous pathway may comprise a depth from about 1 μm to 10 μm. In some embodiments, for instance, the intermediate tortuous pathway may comprise a depth from about 1 μm to about 5 μm. For example, in further embodiments, the intermediate tortuous pathway may comprise a depth of about 3 μm. As such, in certain embodiments, the intermediate tortuous pathway may comprise a depth from at least about any of the following: 1, 1.5, 2, 2.5, 3, 3.5, 4, 4.5, 5, 5.5, 6, 6.5, 7, 7.5, 8, 8.5, 9, 9.5, and 10 μm and/or at most about 10, 9.5, 9, 8.5, 8, 7.5, 7, 6.5, 6, 5.5, 5, 4.5, 4, 3.5, 3, 2.5, 2, 1.5, and 1 μm (e.g., about 2-4 μm, about 3-9 μm, etc.).

In accordance with certain embodiments, for instance, the hydrogel may comprise a natural or synthetic biodegradable polymer. In this regard, the hydrogel may be resorbable by the body. The biodegradable polymer may be a thermoplastic polymer, a thermoset polymer, or any combination thereof. In some embodiments, for example, the thermoset polymer may be crosslinkable. In such embodiments, for instance, the thermoset polymer may be crosslinked using thermal energy and/or irradiation. Irradiation may include ultraviolet light, infrared radiation, microwave radiation, x-rays, electron beam radiation, proton or neutron beam radiation, or a combination thereof. The crosslinked materials can be highly crosslinked or lightly crosslinked in the form of hydrogels.

The biodegradable polymer may include one or more oligomers, homopolymers, a blend or oligomers and/or homopolymers, copolymers, ionomers, polyelectrolytes, dendrimers, or a combination thereof. Copolymers can include block copolymers, random copolymers, gradient copolymers, alternating copolymers, star block copolymers, or combinations thereof.

For example, the biodegradable polymer may comprise one or more polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, styrene acrylonitrile, acrylonitrile-butadiene-styrene (ABS), polyethylene terephthalate, polybutylene terephthalate, polyurethane, ethylene propylene diene rubber (EPR), polytetrafluoroethylene, perfluoroelastomers, fluorinated ethylene propylene, perfluoroalkoxyethylene, polychlorotrifluoroethylene, polyvinylidene fluoride, polysiloxanes, or the like, or a combination thereof.

Examples of polyelectrolytes include polystyrene sulfonic acid, polyacrylic acid, pectin, carrageenan, alginates, carboxymethylcellulose, polyvinylpyrrolidone, or the like, or a combination thereof.

Examples of thermoset polymers include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination thereof.

Examples of blends of thermoplastic polymers include acrylonitrile-butadiene-styrene/nylon, polycarbonate/acrylonitrile-butadiene-styrene, acrylonitrile butadiene styrene/polyvinyl chloride, polyphenylene ether/polystyrene, polyphenylene ether/nylon, polysulfone/acrylonitrile-butadiene-styrene, polycarbonate/thermoplastic urethane, polycarbonate/polyethylene terephthalate, polycarbonate/polybutylene terephthalate, thermoplastic elastomer alloys, nylon/elastomers, polyester/elastomers, polyethylene terephthalate/polybutylene terephthalate, acetal/elastomer, styrene-maleic anhydride/acrylonitrile-butadiene-styrene, polyether etherketone/polyethersulfone, polyether etherketone/polyetherimide polyethylene/nylon, polyethylene/polyacetal, or the like.

According to certain embodiments, for example, the hydrogel may comprise at least one of a gelatin, a collagen, a CAPGEL™ gel, a copper capillary alginate gel (CCAG), a non-CCAG alginate, ethylene glycol dimethacrylate (EGDMA), (hydroxyethyl)methacrylate (HEMA), or any combination thereof. Additional descriptions of suitable hydrogels may be found in PCT/US2016/029122, U.S. Pat. Nos. 8,668,863, 8,946,194, 9,095,558, which are incorporated by reference herein.

In accordance with certain embodiments, for example, the hydrogel may further comprise peptide oligomer chemical patterning. In some embodiments, for instance, the chemical patterning may utilize RAFT polymerization catalysts, as discussed in more detail in PCT/IB2017/053007, which is incorporated by reference in its entirety. In certain embodiments, for example, such chemical patterning may further enable the attachment of poly(L-lysine) (PLL)-terminated grafts and/or non-binding poly(ethylene oxide) (PEO) grafts, as discussed in more detail in PCT/IB2017/053007, which is incorporated by reference in its entirety.

In accordance with certain embodiments, for instance, the hydrogel may further comprise an agent-releasing coating. Such agent-releasing coatings may slowly release integrated molecules that, for example, selectively encourage the growth of sensory nerve fibers and/or encourage the growth of motor nerve fibers. In some embodiments, for instance, a portion of the hydrogel surrounding one set of TEENI threads may include one type of agent-releasing coating, while a separate portion of the hydrogel surrounding a different set of TEENI threads may include a different type of agent-releasing coating depending on the desired outcome for the selected set of TEENI threads.

According to certain embodiments, the agent-releasing coating may include one or more medicaments, vitamins, mineral supplements, substances used for the treatment, prevention, diagnosis, cure or mitigation of disease or illness, substances which affect the structure or function of the body, or drugs. By way of example only, the agent-releasing coating may include, but is not limited to, one or more antibodies, antibody fragments, antibiotics, antifungal agents, antibacterial agents, anti-viral agents, anti-parasitic agents, growth factors, neurotrophic factors, angiogenic factors, anesthetics, mucopolysaccharides, metals, cells, proteins, polynucleotides, polypeptides enzymes, degradation agents, lipids, carbohydrates, chemical compounds such as pharmaceuticals and other wound healing agents. The agent-releasing coating may include therapeutic agents, diagnostic materials, and/or research reagents.

In accordance with certain embodiments, for example, the tissue-engineered nerve scaffold may further comprise one or more tunnels running through the hydrogel. These tunnels may or may not comprise the micropattern. In some embodiments, for instance, the orientation of the tunnels may be varied to produce cell layers having different orientations. By orienting the cells differently, the strength of the new cell layers may be improved. In certain embodiments, for example, the tunnels may extend completely through the hydrogel, but in other embodiments, the tunnels may only extend partially through the hydrogel. In further embodiments, for instance, the tunnels may be surrounded on all sides by the hydrogel, but in other embodiments, one or more surfaces of the tunnels may be an open surface, i.e. the one or more surfaces are open to ambient conditions. According to certain embodiments, the tunnels may be formed by forming the hydrogel around structures that correspond to the shape of the tunnels. These tunnels may include one or more capillaries, pores, micropores, and can have cross-sectional geometries that are square, rectangular, triangular, circular, ellipsoidal, polygonal, or a combination thereof. The channels may have dimensions in the nanometer range or in the micrometer range. By way of example only, the channels may have average cross-sectional dimensions from about 10 nm to about 10 μm.

In accordance with certain embodiments, for example, the neural cells may comprise Schwann cells. In other embodiments, for instance, the neural cells may comprise neural stem cells. Such stem cells may be, for example, embryonic stem cells, somatic stem cells, mesenchymal stem cells, induced pluripotent stem (iPs) cells, and/or the like. In further embodiments, for example, the cells associated with scar-tissue formation and encapsulation may comprise fibroblasts. In some embodiments, for instance, the micropattern may prevent attack by macrophages.

II. Method of Manufacturing a Tissue-Engineered Nerve Scaffold

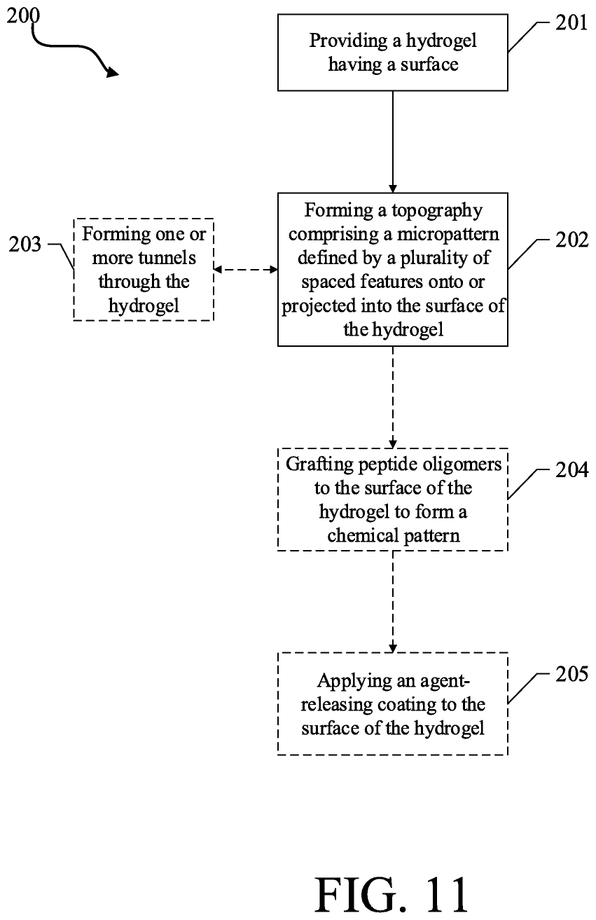

In another aspect, methods of manufacturing tissue-engineered nerve scaffolds are provided. As shown in FIG. 11, the method 200 includes providing a hydrogel having a surface at block 201, forming a topography comprising a micropattern defined by a plurality of spaced features onto or projected into the surface of the hydrogel at block 202, optionally forming one or more tunnels (which may or may not have the micropattern) through the hydrogel at block 203, optionally grafting peptide oligomers to the surface of the hydrogel to form a chemical pattern at block 204, and optionally applying an agent-releasing coating to the surface of the hydrogel at block 205. Each spaced feature is a different length than a neighboring spaced feature. The plurality of spaced features are spaced from each other to define an intermediate tortuous pathway and are arranged in a plurality of groupings. Neighboring groupings share a common feature, and the spaced features within each of the groupings are spaced apart at an average distance from about 10 nm to about 200 μm. In this regard, the micropattern facilitates attachment and alignment of neural cells and reduces attachment and alignment of cells associated with scar-tissue formation and encapsulation.

In accordance with certain embodiments, for instance, providing the hydrogel may comprise providing a hydrogel comprising a natural or synthetic biodegradable polymer, as previously described herein.

In accordance with certain embodiments, for example, forming the topography may comprise embossing the surface of the hydrogel with the micropattern. In other embodiments, for instance, forming the topography may comprise molding the surface of the hydrogel to form the micropattern.

According to certain embodiments, such as those described in detail herein, a single micropatterned hydrogel may surround the multielectrode neural interface. In such embodiments, for example, the method may further comprise bonding the hydrogel directly to one or more electrodes of the multielectrode neural interface.

In other embodiments, however, the micropattern may be formed directly on one or more TEENI electrodes instead of or in addition to the hydrogel. For instance, in some embodiments the micropattern may be formed on the electrode using an etching technique such as, by way of example only, dry reactive ion etching or direct laser etching. In other embodiments, for example, the micropattern may be printed directly on the surface of the electrode using, for example, an add-on manufacturing technique (e.g., 3D printing). In this regard, in certain embodiments, one or more micropatterned electrodes may be surrounded by a patterned or unpatterned hydrogel.

In still further embodiments, the tissue-engineered nerve scaffold may comprise a second micropatterned hydrogel. For example, in some embodiments the first micropatterned hydrogel may be applied to polyimide, and the second micropatterned hydrogel may be cast upon the first micropatterned hydrogel. In this regard, for instance, the method may further comprise casting a second hydrogel on the hydrogel, wherein the second hydrogel comprises a micropattern.

III. Article for Up-Selecting and Down-Selecting Cell Proliferation

In yet another aspect, articles for up-selecting desired cell proliferation and down-selecting undesired cell proliferation are provided. The article includes a surface, the surface of having a topography comprising a micropattern defined by a plurality of spaced features attached to or projected into the article. Each spaced feature is a different length than a neighboring spaced feature. The plurality of spaced features are spaced from each other to define an intermediate tortuous pathway and are arranged in a plurality of groupings. Neighboring groupings share a common feature. In this regard, the micropattern changes cell behavior and differentiates between cell types.

In accordance with certain embodiments, for example, each spaced feature may comprise a uniform width. In some embodiments, for instance, the spaced features within each of the groupings may be spaced apart vertically at a uniform average vertical distance. Similarly, the spaced features within each of the groupings may be spaced apart horizontally at a uniform average horizontal distance. Moreover, the uniform average vertical distance may be equal to the uniform average horizontal distance.

According to certain embodiments, for example, the spaced features within each of the groupings may be spaced apart at an average distance from about 10 nm to about 200 μm. In some embodiments, for instance, each of the groupings may be spaced apart at an average distance from about 10 nm to about 100 μm. In other embodiments, for example, each of the groupings may be spaced apart at an average distance from about 0.5 μm to about 60 μm. In further embodiments, for instance, each of the groupings may be spaced apart at an average distance from about 5 μm to about 60 μm. In certain embodiments, for example, each of the groupings may be spaced apart at an average distance from about 15 μm to about 60 μm.

In accordance with certain embodiments, for instance, the article may comprise a natural or synthetic polymeric material. In some embodiments, for example, the polymeric material may comprise one or more of a thermoplastic polymer and a thermoset polymer. In further embodiments, for instance, the polymeric material may include one or more oligomers, homopolymers, a blend or oligomers and/or homopolymers, copolymers, ionomers, polyelectrolytes, dendrimers, or a combination thereof. Copolymers can include block copolymers, random copolymers, gradient copolymers, alternating copolymers, star block copolymers, or combinations thereof. For example, the biodegradable polymer may comprise one or more polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, styrene acrylonitrile, acrylonitrile-butadiene-styrene (ABS), polyethylene terephthalate, polybutylene terephthalate, polyurethane, ethylene propylene diene rubber (EPR), polytetrafluoroethylene, perfluoroelastomers, fluorinated ethylene propylene, perfluoroalkoxyethylene, polychlorotrifluoroethylene, polyvinylidene fluoride, polysiloxanes, or the like, or a combination thereof. According to certain embodiments, the polymeric material may comprise at least one of polyimide, polydimethylsiloxane, parylene C, amorphous silicon carbide, or any combination thereof.

In accordance with certain embodiments, for example, the article may further comprise peptide oligomer chemical patterning on the surface of the article. Additional discussion of chemical patterning may be found in PCT/IB2017/053007 incorporated by reference in its entirety.

Although neural cells are discussed at detail herein, the article may be used in conjunction with any suitable cell type as understood by one of ordinary skill in the art. By way of example only, the article may be used in conjunction with one or more cell types including, but not limited to, erythrocytes, megakaryocytes, monocytes, connective tissue macrophages, epidermal Langerhans cells, osteoclasts, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells, oogonia/oocytes, spermatids, spermatocytes, spermatogonium cells, spermatozoa, ovarian follicle cells, Sertoli cells, thymus epithelial cells, interstitial kidney cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cells of exocrine glands, ameloblast epithelial cells, planum semilunatum epithelial cells of vestibular system of ear, organ of Corti interdental epithelial cells, loose connective tissue fibroblasts, corneal fibroblasts, tendon fibroblasts, bone marrow reticular tissue fibroblasts, nonepithelial fibroblasts, pericytes, nucleus pulposus cells of intervertebral disc, cementoblasts/cementocytes, odontoblasts, odontocytes, hyaline cartilage chondrocytes, fibrocartilage chondrocytes, elastic cartilage chondrocytes, osteoblasts/osteocytes, osteoprogenitor cells, hyalocytes of vitreous body of eye, stellate cells of perilymphatic space of ear, hepatic stellate cells, pancreatic stelle cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, endothelial cells, kidney parietal cells, kidney glomerulus podocytes, kidney proximal tubule brush border cells, loop of Henle thin segment cells, kidney distal tubule cells, kidney collecting duct cells, adipocytes, liver lipocytes, anterior lens epithelial cells, crystallin-containing lens fiber cells, basket cells, cartwheel cells, stellate cells, Golgi cells, granule cells, Lugaro cells, unipolar brush cells, Martinotti cells, chandelier cells, medium spiny neurons, Cajal-Retzius cells, double-bouquet cells, neurogliaform cells, spinal interneurons, Renshaw cells, spindle neurons, pyramidal cells, astrocytes, ependymal cells, tanycytes, oligodendrocytes, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite glial cells, enteric glial cells, cholinergic neural cells, adrenergic neural cells, peptidergic neural cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells of eye, photoreceptor green-sensitive cone cells of eye, photoreceptor red-sensitive cone cells of eye, proprioceptive primary sensory neurons, touch-sensitive primary sensory neurons, chemoreceptor glomus cells of carotid body cells, outer hair cells of vestibular system of ear, inner hair cells of vestibular system of ears, taste receptor cells of taste bud, surface epithelial cells of stratified squamous epithelia, basal cells of epithelia, urinary epithelium cells, epidermal keratinocytes, epidermal basal cells, keratinocytes of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, somatotropes, lactotropes, thyrotropes, gonadotropes, corticotropes, intermediate pituitary cells, magnocellular neurosecretory cells, gut and respiratory tract cells, thyroid epithelial cells, parafollicular cells, parathyroid chief cells, oxyphil cells, chromaffin cells, adrenal gland cells, Leydig cells of testes, theca interna cells of ovarian follicles, corpus luteum cells, granulosa lutein cells, theca lutein cells, juxtaglomerular cells, macula densa cells of kidney, peripolar cells of kidney, mesangial cells of kidney, alpha cells, beta cells, delta cells, PP cells, epsilon cells, salivary gland mucous cells, salivary gland number 1, Von Ebner's gland cells, mammary gland cells, lacrimal gland cells, ceruminous gland cells, eccrine sweat glandering dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, gland of Littre cells, uterus endometrium cells, insolated goblet cells, stomach lining mucous cells, gastric gland zymogenic cells, gastric gland oxyntic cells, pancreatic acinar cells, Paneth cells, type II pneumocytes, club cells, and/or the like.

In accordance with certain embodiments, for instance, the micropattern (and article) may be configured for at least one of cell isolation, cell selection, inducing selected cellular function, tissue engineering, cell culturing, inducing alignment to induce a selected genotype and phenotype, developing cell lines for screening or evaluation of drug interactions, building and/or 3D printing of viable tissue constructs (e.g., biorobots), or any combination thereof. Similarly, the article may be configured for inclusion in a kit designed to perform any one or more of these applications.

IV. Method of Manufacturing an Article

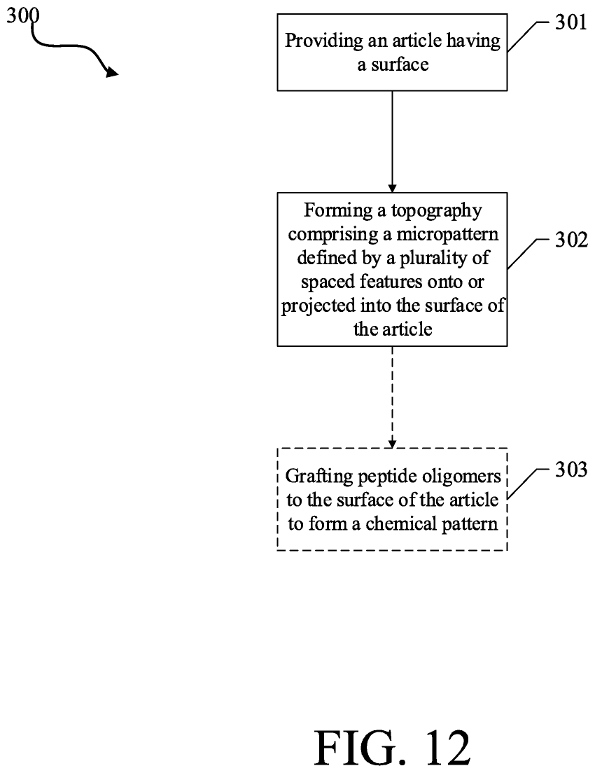

In yet another aspect, methods of manufacturing articles for up-selecting desired cell proliferation and down-selecting undesired cell proliferation are provided. As shown in FIG. 12, the method 300 includes providing an article having a surface at block 301, forming a topography comprising a micropattern defined by a plurality of spaced features onto or projected into the surface of the article at block 302, and optionally grafting peptide oligomers to the surface of the article to form a chemical pattern at block 303. Each spaced feature is a different length than a neighboring spaced feature. The plurality of spaced features are spaced from each other to define an intermediate tortuous pathway and are arranged in a plurality of groupings. Neighboring groupings share a common feature. In this regard, the micropattern changes cell behavior and differentiates between cell types.

In accordance with certain embodiments, for instance, providing the article may comprise providing an article comprising a natural or synthetic polymeric material as previously described herein.

In accordance with certain embodiments, for example, forming the topography may comprise embossing the surface of the article with the micropattern. In other embodiments, for instance, forming the topography may comprise molding the surface of the article to form the micropattern. In further embodiments, for example, the micropattern may be formed on the article using an etching technique such as, by way of example only, dry reactive ion etching or direct laser etching. In other embodiments, for example, the micropattern may be printed directly on the surface of the article using, for example, an add-on manufacturing technique (e.g., 3D printing).

### Example

The following example is provided for illustrating one or more embodiments of the present invention and should not be construed as limiting the invention.

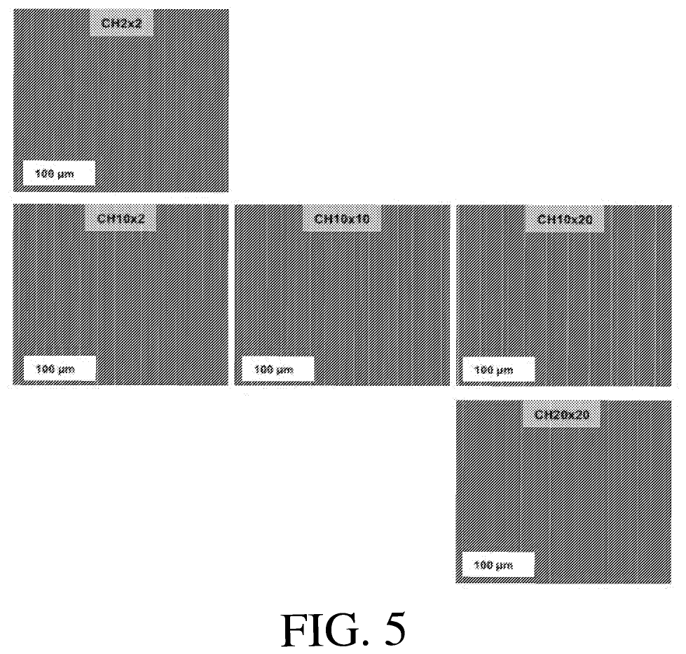

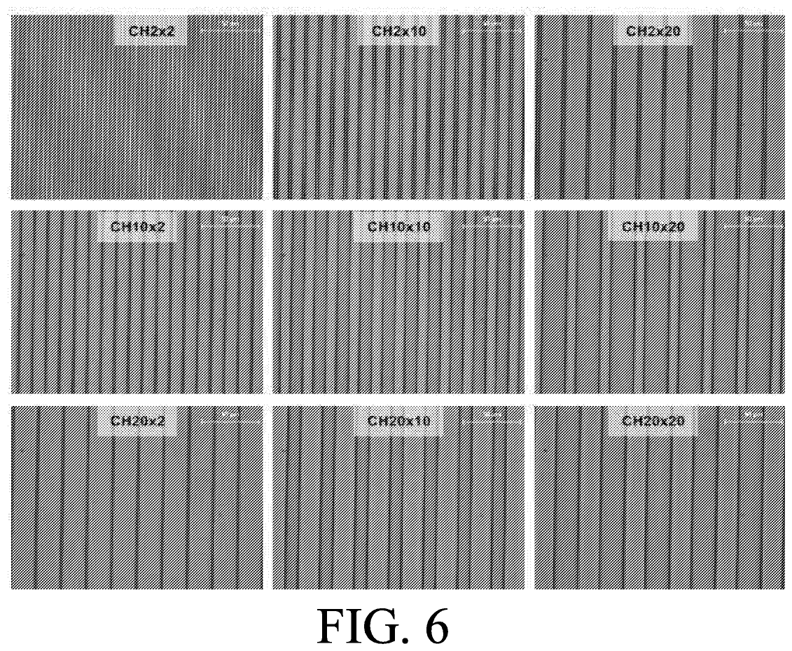

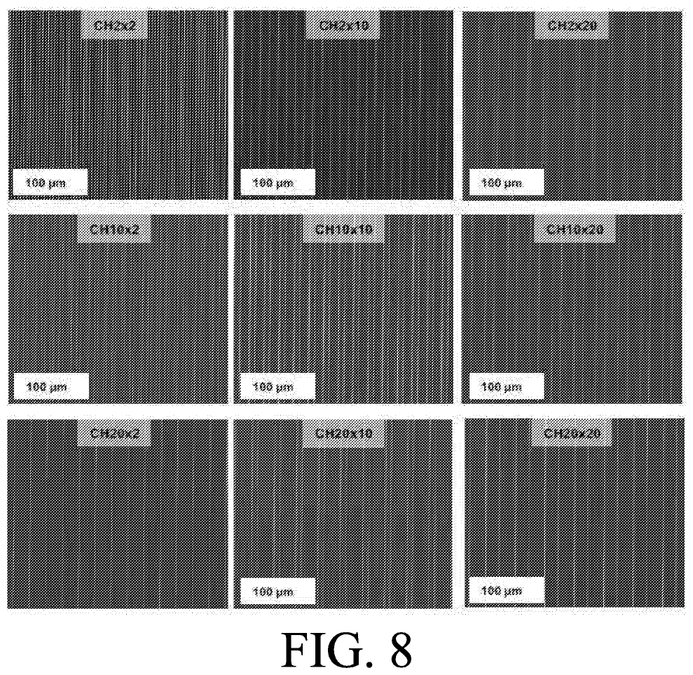

Nineteen different engineered micropatterns including parallel channels (CH) and Sharklet® (SK) were prepared (as shown in FIGS. 3-8) and then screened against fibroblasts and Schwann cells in vitro. Each of the micropatterns were labeled as either CHaxb or SKaxb, where a represents the width of an individual feature, and b represents the horizontal distance between two individual features.

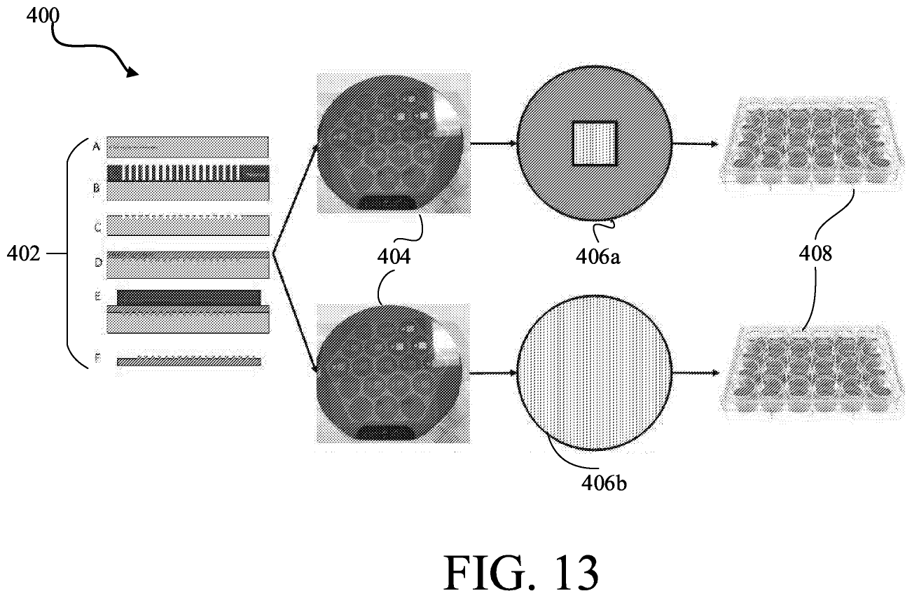

As shown in the preparation method 400 illustrated in FIG. 13, silicon wafers were patterned with 15 mm diameter regions containing the inverse of 19 separate microtopographies with dimensions ranging from 2-20 μm using photolithography and etched up to 3.7 μm deep using deep reactive ion etching. Individual polyimide disks (10 μm thick, 15 mm diameter) were formed by spin coating and curing polyimide (U-Varnish S, UBE Ind.) onto etched wafers and isolated from the cured film by O₂ plasma dry etching, all as shown in step 402. Disks were peeled from the wafer and adhered to 15 mm diameter glass coverslips pattern side up using silicone (RTV 732, Dow Corning), as shown in step 404. Some disks included a patterned region that occupied only a portion of the disk (e.g., 5×5 mm²), as shown in step 406a, while other disks were fully patterned, as shown in step 406b. Smooth polyimide controls were fabricated against a smooth Si wafer in the same fashion. Samples were placed in 24-well cell culture plates, sterilized, and seeded with either rat SCs or fibroblasts at passage 3, as shown in step 408. Cellular viability was assessed and quantified using Alamar Blue Assay at day 1, 3 and 7 and compared to the smooth control (n=3). Actin filaments of the cell cytoskeleton and cell nuclei were stained using Palloidin and DAPI, respectively, and cells were imaged to study the changes of the cell shape and morphology in response to microtopography shape and geometry.

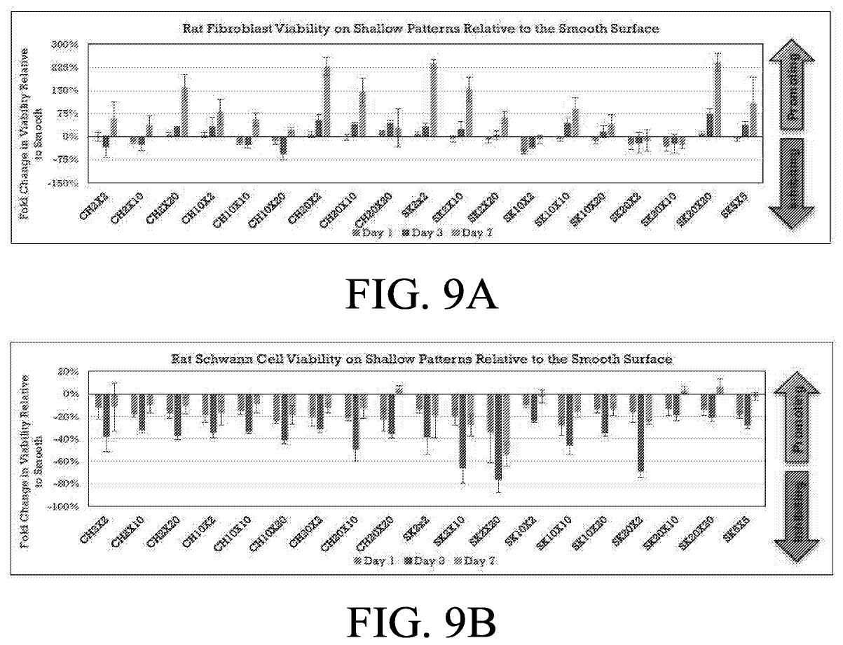

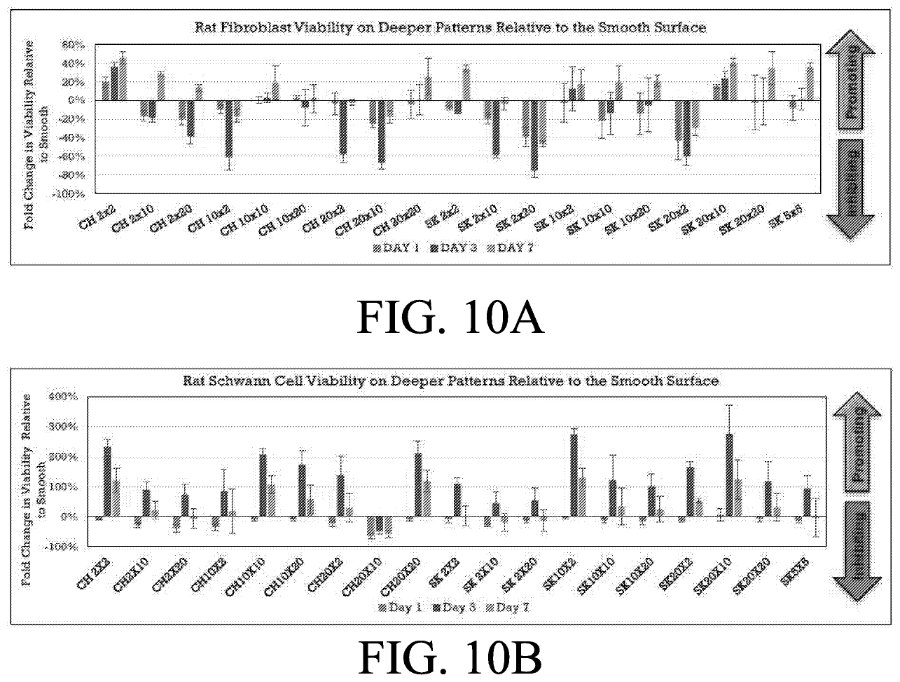

Engineered microtopographies were characterized by profilometry and SEM. Cellular proliferation was normalized against smooth controls, and as shown in FIGS. 9A-10B, results show a strong correlation between pattern dimension and geometry with a mix of inhibitory and promoting patterns. For example, it was found that the Sharklet micropatterns, particularly SK20×2, inhibited fibroblast adhesion and proliferation while promoting Schwann cell proliferation. In particular, as shown in FIGS. 9A-10B, the deeper SK20×2 micropattern (i.e. having a 3 μm depth compared to a 1.3 μm depth) inhibited fibroblast adhesion and proliferation while promoting Schwann cell proliferation better than all other CH and SK micropatterns at either depth.

### NON-LIMITING EXEMPLARY EMBODIMENTS

Having described various aspects and embodiments of the invention herein, further specific embodiments of the invention include those set forth in the following paragraphs.

Certain embodiments provide tissue-engineered nerve scaffolds, methods of manufacturing tissue-engineered nerve scaffolds, articles for up-selecting desired cell proliferation and down-selecting undesired cell proliferation, and methods of manufacturing articles for up-selecting desired cell proliferation and down-selecting undesired cell proliferation. In one aspect, a tissue-engineered nerve scaffold configured to coat a multielectrode neural interface is provided. The tissue-engineered nerve scaffold may include a hydrogel having a surface. The surface may have a topography comprising a micropattern defined by a plurality of spaced features attached to or projected into the hydrogel. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature, and the spaced features within each of the groupings may be spaced apart as an average distance from about 10 nm to about 200 μm. The micropattern facilitates attachment and alignment of neural cells and reduces attachment and alignment of cells associated with scar-tissue formation and encapsulation.

In accordance with certain embodiments, for example, each spaced feature may comprise a uniform width. In some embodiments, for instance, the spaced features within each of the groupings may be spaced apart vertically at a uniform average vertical distance, and the spaced features within each of the groupings may be spaced apart horizontally at a uniform average horizontal distance. In further embodiments, for example, the uniform average vertical distance may be equal to the uniform average horizontal distance.

According to certain embodiments, for instance, each spaced feature may comprise a uniform width from about 2 μm to about 20 μm. In some embodiments, for example, the spaced features within each of the groupings may be spaced apart at a uniform average vertical distance from about 2 μm to about 20 μm. Similarly, the spaced features within each of the groupings may be spaced apart at a uniform average horizontal distance from about 2 μm to about 20 μm. In further embodiments, for instance, each spaced feature may comprise a uniform width of about 20 μm, and the spaced features within each of the groupings may be spaced apart at a uniform average horizontal distance and a uniform average vertical distance of about 2 μm.

In accordance with certain embodiments, for example, the intermediate tortuous pathway may comprise a depth from about 1 μm to about 10 μm. In further embodiments, for instance, the intermediate tortuous pathway may comprise a depth of about 3 μm.

In accordance with certain embodiments, for example, the hydrogel may comprise a natural or synthetic biodegradable polymer.

In accordance with certain embodiments, for instance, the hydrogel may be bonded directly to one or more electrodes of the multielectrode neural interface. In some embodiments, for example, one or more electrodes of the multielectrode neural interface may comprise a topography having a micropattern defined by a plurality of spaced features attached to or projected into a surface of the one or more electrodes. In further embodiments, for instance, the tissue-engineered nerve scaffold may further comprise a second hydrogel having a micropattern.

In accordance with certain embodiments, for example, the hydrogel may further comprise peptide oligomer chemical patterning. In some embodiments, for instance, the hydrogel may further comprise an agent-releasing coating. In further embodiments, for example, the tissue-engineered nerve scaffold may further comprise one or more tunnels running through the hydrogel.

In accordance with certain embodiments, for instance, the neural cells may comprise Schwann cells. In other embodiments, for example, the neural cells may comprise neural stem cells. In some embodiments, for instance, the cells associated with scar-tissue formation and encapsulation may comprise fibroblasts. In further embodiments, for example, the micropattern may prevent attack by macrophages.

In another aspect, a method of manufacturing a tissue-engineered nerve scaffold configured to coat a multielectrode neural interface is provided. The method may include providing a hydrogel having a surface and forming a topography comprising a micropattern defined by a plurality of spaced features onto or projected into the surface of the hydrogel. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature, and the spaced features within each of the groupings may be spaced apart as an average distance from about 10 nm to about 200 μm. The micropattern facilitates attachment and alignment of neural cells and reduces attachment and alignment of cells associated with scar-tissue formation and encapsulation.

In accordance with certain embodiments, for example, providing the hydrogel may comprise providing a hydrogel comprising a natural or synthetic biodegradable polymer.

In accordance with certain embodiments, for instance, forming the topography may comprise embossing the surface of the hydrogel with the micropattern. In other embodiments, for example, forming the topography may comprise molding the surface of the hydrogel to form the micropattern.

In accordance with certain embodiments, for instance, the method may further comprise bonding the hydrogel directly to one or more electrodes of the multielectrode neural interface. In some embodiments, for example, the method may further comprise grafting peptide oligomers to the surface of the hydrogel to form a chemical pattern. In further embodiments, for instance, the method may further comprise applying an agent-releasing coating to the surface of the hydrogel. In certain embodiments, for example, the method may further comprise forming one or more tunnels through the hydrogel. In some embodiments, for instance, the method may further comprise forming a topography comprising a micropattern defined by a plurality of spaced features onto or projected into a surface of one or more electrodes of the multielectrode neural interface. In further embodiments, for example, the method may further comprise casting a second hydrogel on the hydrogel, wherein the second hydrogel comprises a micropattern.

In accordance with certain embodiments, for example, each spaced feature may comprise a uniform width. In some embodiments, for instance, the spaced features within each of the groupings may be spaced apart vertically at a uniform average vertical distance, and the spaced features within each of the groupings may be spaced apart horizontally at a uniform average horizontal distance. In further embodiments, for example, the uniform average vertical distance may be equal to the uniform average horizontal distance.

According to certain embodiments, for instance, each spaced feature may comprise a uniform width from about 2 μm to about 20 μm. In some embodiments, for example, the spaced features within each of the groupings may be spaced apart at a uniform average vertical distance from about 2 μm to about 20 μm. Similarly, the spaced features within each of the groupings may be spaced apart at a uniform average horizontal distance from about 2 μm to about 20 μm. In further embodiments, for instance, each spaced feature may comprise a uniform width of about 20 μm, and the spaced features within each of the groupings may be spaced apart at a uniform average horizontal distance and a uniform average vertical distance of about 2 μm.

In accordance with certain embodiments, for example, the intermediate tortuous pathway may comprise a depth from about 1 μm to about 10 μm. In further embodiments, for instance, the intermediate tortuous pathway may comprise a depth of about 3 μm.

In accordance with certain embodiments, for instance, the neural cells may comprise Schwann cells. In other embodiments, for example, the neural cells may comprise neural stem cells. In some embodiments, for instance, the cells associated with scar-tissue formation and encapsulation may comprise fibroblasts. In further embodiments, for example, the micropattern may prevent attack by macrophages.

In yet another aspect, an article for up-selecting desired cell proliferation and down-selecting undesired cell proliferation is provided. The article may include a surface. The surface may have a topography comprising a micropattern defined by a plurality of spaced features attached to or projected into the article. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature. The micropattern changes cell behavior and differentiates between cell types.

In accordance with certain embodiments, for example, each spaced feature may comprise a uniform width. In some embodiments, for instance, the spaced features within each of the groupings may be spaced apart vertically at a uniform average vertical distance, and the spaced features within each of the groupings may be spaced apart horizontally at a uniform average horizontal distance. In further embodiments, for example, the uniform average vertical distance may be equal to the uniform average horizontal distance. In certain embodiments, for instance, the spaced features within each of the groupings may be spaced apart at an average distance from about 10 nm to about 200 μm.

In accordance with certain embodiments, for example, the article may comprise a natural or synthetic polymeric material. In some embodiments, for instance, the article may further comprise peptide oligomer chemical patterning on the surface of the article.

In accordance with certain embodiments, for example, the micropattern may be configured for at least one of cell isolation cell selection, inducing selected cellular function, tissue engineering, cell culturing, inducing alignment to induce a selected genotype and phenotype, developing cell lines for screening or evaluation of drug interactions, building of viable tissue constructs, or any combination thereof.

In still yet another aspect, a method of manufacturing an article having a surface is provided. The method may include providing the article and forming a topography comprising a micropattern defined by a plurality of spaced features onto or projected into the surface of the article. Each spaced feature may be a different length than a neighboring spaced feature. The plurality of spaced features may be spaced from each other to define an intermediate tortuous pathway. The plurality of spaced features may be arranged in a plurality of groupings such that neighboring groupings share a common feature. The micropattern changes cell behavior and differentiates between cell types.

In accordance with certain embodiments, for example, providing the article may comprise providing an article comprising a natural or synthetic polymeric material.

In accordance with certain embodiments, for instance, forming the topography may comprise embossing the surface of the article with the micropattern. In other embodiments, for example, forming the topography may comprise molding the surface of the article to form the micropattern. In some embodiments, for instance, the method may further comprise grafting peptide oligomers to the surface of the article to form a chemical pattern.

In accordance with certain embodiments, for example, each spaced feature may comprise a uniform width. In some embodiments, for instance, the spaced features within each of the groupings may be spaced apart vertically at a uniform average vertical distance, and the spaced features within each of the groupings may be spaced apart horizontally at a uniform average horizontal distance. In further embodiments, for example, the uniform average vertical distance may be equal to the uniform average horizontal distance.

In accordance with certain embodiments, for example, the micropattern may be configured for at least one of cell isolation cell selection, inducing selected cellular function, tissue engineering, cell culturing, inducing alignment to induce a selected genotype and phenotype, developing cell lines for screening or evaluation of drug interactions, building of viable tissue constructs, or any combination thereof.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A tissue-engineered nerve scaffold configured to coat a multielectrode neural interface, the tissue-engineered nerve scaffold comprising a hydrogel having a surface, the surface having a topography comprising a micropattern defined by a plurality of spaced features attached to or projected into the hydrogel,
wherein each spaced feature is a different length than a neighboring spaced feature, the plurality of spaced features are spaced from each other to define an intermediate tortuous pathway, the plurality of spaced features are arranged in a plurality of groupings, neighboring groupings share a common feature, and the spaced features within each of the groupings are spaced apart at an average distance from about 10 nm to about 200 μm; and
wherein the micropattern facilitates attachment and alignment of neural cells and reduces attachment and alignment of cells associated with scar-tissue formation and encapsulation.

2. The tissue-engineered nerve scaffold according to claim 1, wherein:
each spaced feature comprises a uniform width;
the spaced features within each of the groupings are spaced apart vertically at a uniform average vertical distance;
the spaced features within each of the groupings are spaced apart horizontally at a uniform average horizontal distance; and
the uniform average vertical distance is equal to the uniform average horizontal distance.

3. The tissue-engineered nerve scaffold according to claim 1, wherein each spaced feature comprises a uniform width from about 2 μm to about 20 μm.

4. The tissue-engineered nerve scaffold according to claim 1, wherein the spaced features within each of the groupings are spaced apart at a uniform average vertical distance from about 2 μm to about 20 μm.

5. The tissue-engineered nerve scaffold according to claim 1, wherein the spaced features within each of the groupings are spaced apart at a uniform average horizontal distance from about 2 μm to about 20 μm.

6. The tissue-engineered nerve scaffold according to claim 1, wherein each spaced feature comprises a uniform width of about 20 μm, and the spaced features within each of the groupings are spaced apart at a uniform average horizontal distance and a uniform average vertical distance of about 2 μm.

7. The tissue-engineered nerve scaffold according claim 1, wherein the intermediate tortuous pathway comprises a depth from about 1 μm to about 10 μm.

8. The tissue-engineered nerve scaffold according to claim 1, wherein the intermediate tortuous pathway comprises a depth of about 3 μm.

9. The tissue-engineered nerve scaffold according to claim 1, wherein the hydrogel comprises a natural or synthetic biodegradable polymer.

10. The tissue-engineered nerve scaffold according to claim 1, wherein the hydrogel is bonded directly to one or more electrodes of the multielectrode neural interface.

11. The tissue-engineered nerve scaffold according to claim 1, wherein the hydrogel further comprises peptide oligomer chemical patterning.

12. The tissue-engineered nerve scaffold according to claim 1, wherein the hydrogel further comprises an agent-releasing coating.

13. The tissue-engineered nerve scaffold according to claim 1, wherein one or more electrodes of the multielectrode neural interface comprises a topography having a micropattern defined by a plurality of spaced features attached to or projected into a surface of the one or more electrodes.

14. The tissue-engineered nerve scaffold according to claim 1, further comprising a second hydrogel having a micropattern.

15. The tissue-engineered nerve scaffold according to claim 1, further comprising one or more tunnels running through the hydrogel.

16. The tissue-engineered nerve scaffold according to claim 1, wherein the neural cells comprise Schwann cells.

17. The tissue-engineered nerve scaffold according to claim 1, wherein the neural cells comprise neural stem cells.

18. The tissue-engineered nerve scaffold according to claim 1, wherein the cells associated with scar-tissue formation and encapsulation comprise fibroblasts.

19. The tissue-engineered nerve scaffold according to claim 1, wherein the micropattern prevents attack by macrophages.

20. A method of manufacturing a tissue-engineered nerve scaffold configured to coat a multielectrode neural interface, the method comprising:
providing a hydrogel having a surface; and
forming a topography comprising a micropattern defined by a plurality of spaced features onto or projected into the surface of the hydrogel,
wherein each spaced feature is a different length than a neighboring spaced feature, the plurality of spaced features are spaced from each other to define an intermediate tortuous pathway, the plurality of spaced features are arranged in a plurality of groupings, neighboring groupings share a common feature, and the spaced features within each of the groupings are spaced apart at an average distance from about 10 nm to about 200 μm; and
wherein the micropattern facilitates attachment and alignment of neural cells and reduces attachment and alignment of cells associated with scar-tissue formation and encapsulation.

21. The method according to claim 20, wherein providing the hydrogel comprises providing a hydrogel comprising a natural or synthetic biodegradable polymer.

22. The method according to claim 20, wherein forming the topography comprises embossing the surface of the hydrogel with the micropattern.

23. The method according to claim 20, wherein forming the topography comprises molding the surface of the hydrogel to form the micropattern.

24. The method according to claim 20, further comprising bonding the hydrogel directly to one or more electrodes of the multielectrode neural interface.

25. The method according to claim 20, further comprising grafting peptide oligomers to the surface of the hydrogel to form a chemical pattern.

26. The method according to claim 20, further comprising applying an agent-releasing coating to the surface of the hydrogel.

27. The method according to claim 20, further comprising forming one or more tunnels through the hydrogel.

28. The method according to claim 20, further comprising forming a topography comprising a micropattern defined by a plurality of spaced features onto or projected into a surface of one or more electrodes of the multielectrode neural interface.

29. The method according to claim 20, further comprising casting a second hydrogel on the hydrogel, wherein the second hydrogel comprises a micropattern.

30. The method according to claim 20, wherein:
each spaced feature comprises a uniform width;
the spaced features within each of the groupings are spaced apart vertically at a uniform average vertical distance;
the spaced features within each of the groupings are spaced apart horizontally at a uniform average horizontal distance; and
the uniform average vertical distance is equal to the uniform average horizontal distance.

31. The method according to claim 20, wherein each spaced feature comprises a uniform width from about 2 μm to about 20 μm.

32. The method according to claim 20, wherein the spaced features within each of the groupings are spaced apart at a uniform average vertical distance from about 2 μm to about 20 μm.

33. The method according to claim 20, wherein the spaced features within each of the groupings are spaced apart at a uniform average horizontal distance from about 2 μm to about 20 μm.

34. The method according to claim 20, wherein each spaced feature comprises a uniform width of about 20 μm, and the spaced features within each of the groupings are spaced apart at a uniform average horizontal distance and a uniform average vertical distance of about 2 μm.

35. The method according to claim 20, wherein the intermediate tortuous pathway comprises a depth from about 1 μm to about 10 μm.

36. The method according to claim 20, wherein the intermediate tortuous pathway comprises a depth of about 3 μm.

37. The method according to claim 20, wherein the neural cells comprise Schwann cells.

38. The method according to claim 20, wherein the neural cells comprise neural stem cells.

39. The method according to claim 20, wherein the cells associated with scar-tissue formation and encapsulation comprise fibroblasts.

40. The method according to claim 20, wherein the micropattern prevents attack by macrophages.

41. An article for up-selecting desired cell proliferation and down-selecting undesired cell proliferation, the article comprising a surface, the surface having a topography comprising a micropattern defined by a plurality of spaced features attached to or projected into the article,
wherein each spaced feature is a different length than a neighboring spaced feature, the plurality of spaced features are spaced from each other to define an intermediate tortuous pathway, the plurality of spaced features are arranged in a plurality of groupings, and neighboring groupings share a common feature; and
wherein the micropattern changes cell behavior and differentiates between cell types.

42. The article according to claim 41, wherein:
each spaced feature comprises a uniform width;
the spaced features within each of the groupings are spaced apart vertically at a uniform average vertical distance;
the spaced features within each of the groupings are spaced apart horizontally at a uniform average horizontal distance; and
the uniform average vertical distance is equal to the uniform average horizontal distance.

43. The article according to claim 41, wherein the spaced features within each of the groupings are spaced apart at an average distance from about 10 nm to about 200 μm.

44. The article according to claim 41, wherein the article comprises a natural or synthetic polymeric material.

45. The article according to claim 41, further comprising peptide oligomer chemical patterning on the surface of the article.

46. The article according to claim 41, wherein the micropattern is configured for at least one of cell isolation cell selection, inducing selected cellular function, tissue engineering, cell culturing, inducing alignment to induce a selected genotype and phenotype, developing cell lines for screening or evaluation of drug interactions, building of viable tissue constructs, or any combination thereof.

47. A method of manufacturing an article having a surface, the method comprising:
providing the article; and
forming a topography comprising a micropattern defined by a plurality of spaced features onto or projected into the surface of the article,
wherein each spaced feature is a different length than a neighboring spaced feature, the plurality of spaced features are spaced from each other to define an intermediate tortuous pathway, the plurality of spaced features are arranged in a plurality of groupings, and neighboring groupings share a common feature; and
wherein the micropattern changes cell behavior and differentiates between cell types.

48. The method according to claim 47, wherein providing the article comprises providing an article comprising a natural or synthetic polymeric material.

49. The method according to claim 47, wherein forming the topography comprises embossing the surface of the article with the micropattern.

50. The method according to claim 47, wherein forming the topography comprises molding the surface of the article to form the micropattern.

51. The method according to claim 47, further comprising grafting peptide oligomers to the surface of the article to form a chemical pattern.

52. The method according to claim 47, wherein:
each spaced feature comprises a uniform width;
the spaced features within each of the groupings are spaced apart vertically at a uniform average vertical distance;
the spaced features within each of the groupings are spaced apart horizontally at a uniform average horizontal distance; and
the uniform average vertical distance is equal to the uniform average horizontal distance.

53. The method according to claim 47, wherein the micropattern is configured for at least one of cell selection, inducing selected cellular function, tissue engineering, cell culturing, inducing alignment to induce a selected genotype and phenotype, developing cell lines for screening or evaluation of drug interactions, building of viable tissue constructs, or any combination thereof.
